(19) United States
(12) Patent Application Publication
(10) Pub. No.: US 2005/0037056 A1
(43) Pub. Date: Feb. 17, 2005

(54) Health insole

(75) Inventor: Shou-I Su, Hsichich City (TW)

Correspondence Address: Dougherty & Troxell, ONE SKYLINE PLACE, 5205 LEESBURG PIKE, SUITE 1404, FALLS CHURCH, VA, 22041, US

(73) Assignee: Foto Star-Tech Co., Ltd.

(21) Appl. No.: 10/934,539

(22) Filed: Sep. 7, 2004

(30) Foreign Application Priority Data
Aug. 15, 2003 (TW) 092214833

(51) Int. Cl. A61K 9/70; A43B 13/38

(52) U.S. Cl. 424/443; 36/43

(57) ABSTRACT

A health insole includes a top photocatalyst layer of high performance fibers containing a photocatalyst material, and a bottom functional layer of nonwoven fabric treated with active carbon/far infrared light/fragrance and bonded to the top photocatalyst layer.

### BACKGROUND OF THE INVENTION

1. Field of the Invention

The present invention relates generally to an insole for shoe and more particularly, to a health insole that removes odor and kill germs.

2. Description of the Related Art

Conventionally, a shoe is simply an outer covering for a person's foot. However, the inside space of a shoe tends to be covered with dirt that invites germs. Because of bad ventilation, a shoe may produce an odor when removed from the foot. In order to remove odor and kill germs, various health insole and materials for insole are developed. Exemplars are seen as follows:

1. Japanese Patent No. 1-280073 discloses a cotton fabric coated with a coating material that absorbs moisture and remove odor. Because the coating material is directly coated on the cotton fabric, the finished product is not soft enough.

2. Japanese Patent No. 2-63403 discloses an insole that removes odor. The insole is made of polymer fibers prepared from RCOOH and metal salt. During fabrication, the odor removable substance is directly added to polymer to making fibers. In order to improve tensile strength and friction force, the synthetic fibers are immersed in elastic polymer. Because the fibers are covered by elastic polymer, the odor removing power is lowered.

3. Japanese Patent No. 11-100778 discloses an absorptive synthetic leather containing a photocatalyst suitable for making lining and insole for shoe. This structure of absorptive synthetic leather is a single layer design prepared from different kinds of fibers. Because of a single layer design, the odor removing power is low. Further, the fabrication procedure of this structure of absorptive synthetic leather is complicated.

4. Japanese Utility No. 3086842 discloses a size-adjustable inexpensive insole that removes odor and kills germs. This structure of insole comprises a first layer of paper containing a photocatalyst material and having raised and sink lines, and a second layer of paper substrate having pores. When radiating the first layer of photocatalyst material with ultraviolet light, a photocatalytic reaction is produced, thereby causing the photocatalyst material to release active free ion radical. Because photocatalyst material is added to paper to form the first layer of paper containing a photocatalyst material, this structure of insole does not produce a satisfactory photocatalytic effect. Because this structure of insole is mainly made of paper, it is not durable in use. Further, the raised and sink lines of the first layer of paper containing a photocatalyst material tends to be covered with dust, and it is difficult to clean the first layer of paper containing a photocatalyst material.

5. Japanese Utility No. 3054912 discloses an insole that removes odor and dirt and kills germs. This insole has five-layer structure comprising a first layer of polyester fibers, a second layer of cushion material, a third layer of absorptive sheet material, a fourth layer of titanium dioxide photocatalyst material, and a fifth layer of base material. Because the layer of titanium dioxide photocatalyst material is at the fourth layer, the photocatalytic effect of this structure insole is low.

### SUMMARY OF THE INVENTION

The present invention has been accomplished under the circumstances in view. According to one embodiment of the present invention, the health insole comprises a top photocatalyst layer and a bottom functional layer. The bottom functional layer has a top surface bonded to the top photocatalyst layer. The photocatalyst layer is a layer of high performance fibers containing a photocatalyst material. The functional layer is a layer of nonwoven fabric treated with active carbon, far infrared light, or fragrance essence. According to another embodiment of the present invention, the health insole further comprises a bottom photocatalyst layer bonded to the bottom surface of the bottom functional layer.

### BRIEF DESCRIPTION OF THE DRAWINGS

FIG. 1 is a sectional view of a health insole according to the first embodiment of the present invention.

FIG. 2a is a sectional view of a health insole according to the second embodiment of the present invention.

FIG. 2b is a sectional view of a health insole according to the third embodiment of the present invention.

FIG. 2c is a sectional view of a health insole according to the fourth embodiment of the present invention.

FIG. 3a is a sectional view of a health insole according to the fifth embodiment of the present invention.

FIG. 3b is a sectional view of a health insole according to the sixth embodiment of the present invention.

FIG. 3c is a sectional view of a health insole according to the seventh embodiment of the present invention.

FIG. 4a is a sectional view of a health insole according to the eighth embodiment of the present invention.

FIG. 4b is a sectional view of a health insole according to the ninth embodiment of the present invention.

FIG. 4c is a sectional view of a health insole according to the tenth embodiment of the present invention.

FIG. 5 is a sectional view of a health insole according to the eleventh embodiment of the present invention.

### DETAILED DESCRIPTION OF THE INVENTION

Referring to FIGS. 12c, a health insole has shown comprising a photocatalyst layer 1 and at least one functional layer 2.

The photocatalyst layer 1 by coating, impregnating to apply nanometer photocatalyst material (for example, titanium dioxide). Alternatively, the photocatalyst layer 1 can be commercially available photocatalyst-contained high performance fibers (micro fibers) such as nature fibers, corn fibers, acrylic fibers, nylon 6, nylon 66, high-strength titanium dioxide fibers, or visible light-reactive photocatalyst paper, cloth, leather or synthetic resin. When radiated by ultraviolet light, the photocatalyst layer 1 provides the functions of removing odor and killing germs. Furthermore, by coating or impregnating to apply the photocatalyst material, the physical properties of the photocatalyst layer 1 could be improved to be much more softer.

The functional layer 2 is bonded to the bottom surface of the photocatalyst layer 1 to provide the function of adsorbing organic matter, absorbing moisture, keeping warm, or removing odor.

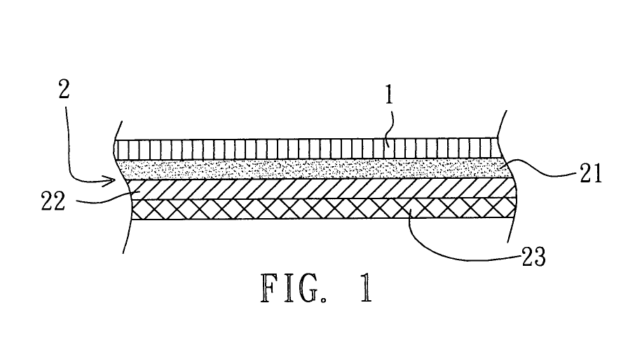

FIG. 1 shows a first embodiment of the present invention. According to this embodiment, the functional layer 2 comprises an inner layer of active carbon-coated nonwoven fabric 21 bonded to the bottom surface of the photocatalyst layer 1, an outer layer of fragrance-treated nonwoven fabric 23, and an intermediate layer of far infrared light-processed nonwoven fabric 22 sandwiched in between the inner layer of active carbon-coated nonwoven fabric 21 and the outer layer of fragrance-treated nonwoven fabric 23. The intermediate layer of far infrared light-processed nonwoven fabric 22 reflects radiating heat of the body, thereby reducing dissipation of heat energy of the body and keeping the body warm. Therefore, the intermediate layer of far infrared light-processed nonwoven fabric 22 improves metabolism and accelerates blood circulation.

The outer layer of fragrance-treated nonwoven fabric 23 provides a good smell to remove odor.

Further, the material for the aforesaid nonwoven fabric can be obtained from paper, fabric, leather, or synthetic resin.

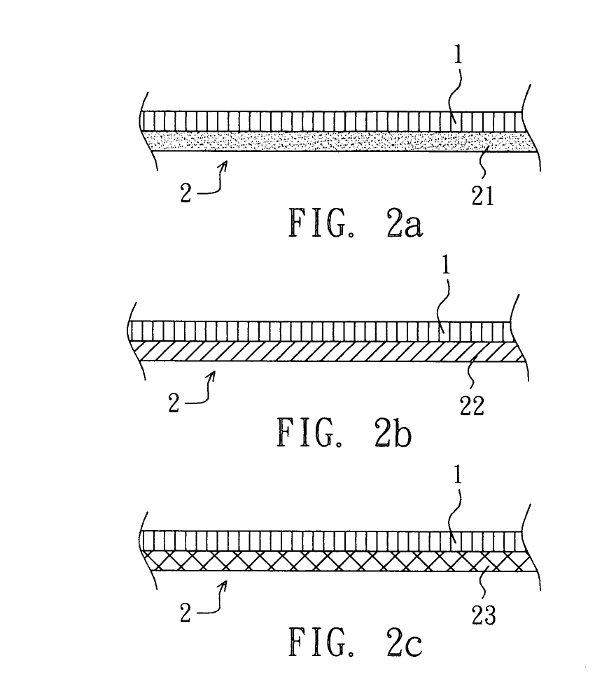

FIG. 2a shows a second embodiment of the present invention. According to this embodiment, the health insole is comprised of a photocatalyst layer 1 and a layer of active carbon-coated nonwoven fabric 21 bonded to the bottom surface of the photocatalyst layer 1.

FIG. 2b shows a third embodiment of the present invention. According to this embodiment, the health insole is comprised of a photocatalyst layer 1 and a layer of fragrance-treated nonwoven fabric 23 bonded to the bottom surface of the photocatalyst layer 1.

FIG. 2c shows a fourth embodiment of the present invention. According to this embodiment, the health insole is comprised of a photocatalyst layer 1 and a layer of far infrared light-processed nonwoven fabric 22 bonded to the bottom surface of the photocatalyst layer 1.

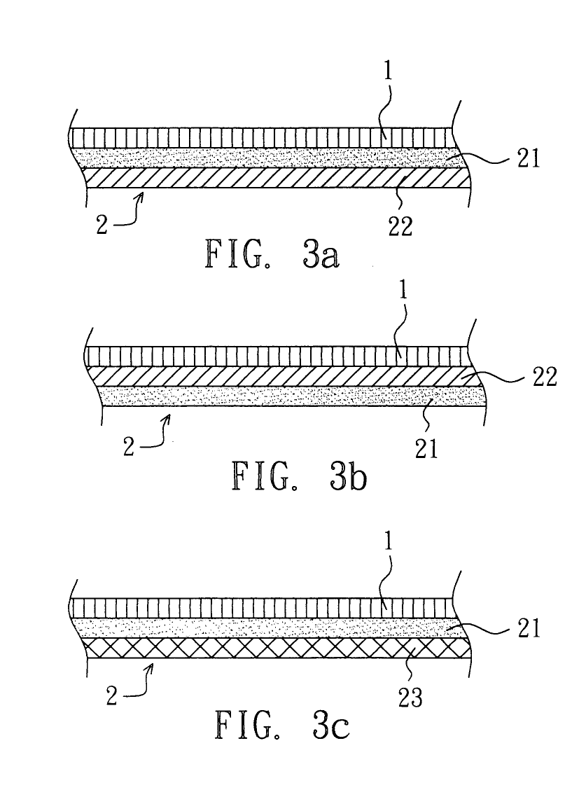

FIG. 3a shows a fifth embodiment of the present invention. According to this embodiment, the health insole is comprised of a photocatalyst layer 1, a layer of active carbon-coated nonwoven fabric 21, and a layer of far infrared light-processed nonwoven fabric 22 sandwiched in between the bottom surface of the photocatalyst layer 1 and the layer of active carbon-coated nonwoven fabric 21.

FIG. 3b shows a sixth embodiment of the present invention. According to this embodiment, the health insole is comprised of a photocatalyst layer 1, a layer of active carbon-coated nonwoven fabric 21, and a layer of far infrared light-processed nonwoven fabric 22 sandwiched in between the bottom surface of the photocatalyst layer 1 and the layer of active carbon-coated nonwoven fabric 21.

FIG. 3c shows a seventh embodiment of the present invention. According to this embodiment, the health insole is comprised of a photocatalyst layer 1, a layer of fragrance-treated nonwoven fabric 23, and a layer of active carbon-coated nonwoven fabric 21 sandwiched in between the bottom surface of the photocatalyst layer 1 and the and a layer of fragrance-treated nonwoven fabric 23.

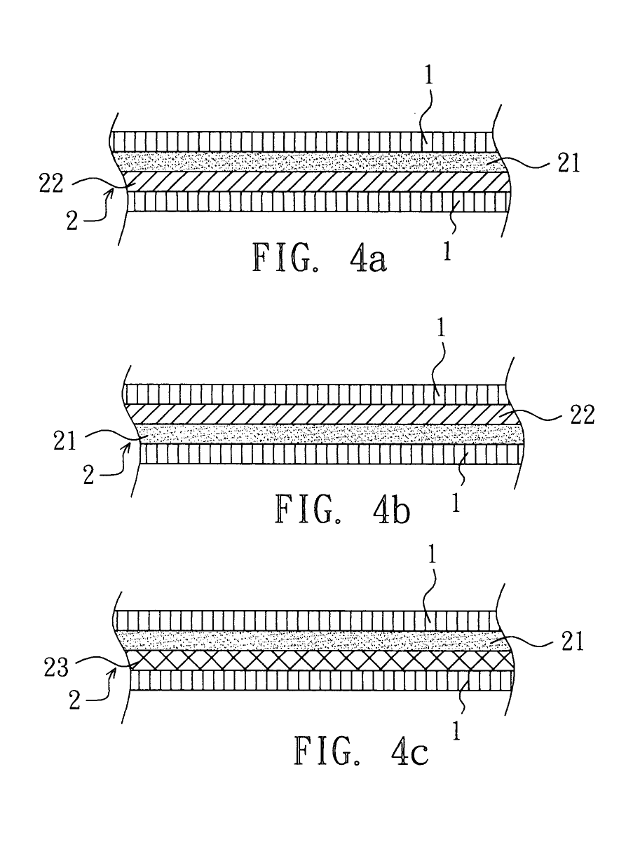

FIG. 4a shows an eighth embodiment of the present invention. According to this embodiment, the health insole is comprised of two photocatalyst layers 1, and a functional layer 2 sandwiched in between the photocatalyst layers 1, wherein the functional layer 2 is comprised of a layer of active carbon-coated nonwoven fabric 21 and a layer of far infrared light-processed nonwoven fabric 22.

FIG. 4b shows a ninth embodiment of the present invention. This embodiment is substantially similar to the aforesaid seventh embodiment with the exception of that the layer of far infrared light-processed nonwoven fabric 22 is bonded to the top surface of the layer of active carbon-coated nonwoven fabric 21 and a.

FIG. 4c shows a tenth embodiment of the present invention. According to this embodiment, the health insole is comprised of two photocatalyst layers 1, and a functional layer 2 sandwiched in between the photocatalyst layers 1, wherein the functional layer 2 is comprised of a layer of active carbon-coated nonwoven fabric 21 and a layer of fragrance-treated nonwoven fabric 23.

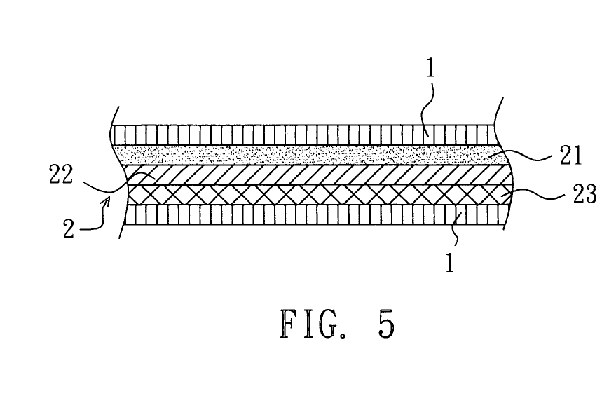

FIG. 5 shows an eleventh embodiment of the present invention. According to this embodiment, the health insole is comprised of two photocatalyst layers 1, and a functional layer 2 sandwiched in between the photocatalyst layers 1, wherein the functional layer 2 is comprised of a layer of active carbon-coated nonwoven fabric 21 and a layer of fragrance-treated nonwoven fabric 23 respectively bonded to the photocatalyst layers 1, and a layer of far infrared light-processed nonwoven fabric 22 sandwiched in between the layer of active carbon-coated nonwoven fabric 21 and the layer of fragrance-treated nonwoven fabric 23.

A prototype of health insole has been constructed with the features of FIGS. 1˜5. The health insole functions smoothly to provide all of the features discussed earlier.

Although particular embodiments of the invention have been described in detail for purposes of illustration, various modifications and enhancements may be made without departing from the spirit and scope of the invention. Accordingly, the invention is not to be limited except as by the appended claims.

1. A health insole comprising a top photocatalyst layer and a bottom functional layer, said bottom functional layer having a top surface bonded to said top photocatalyst layer and a bottom surface, said photocatalyst layer being a layer of high performance fibers containing a photocatalyst material, said functional layer being a layer of nonwoven fabric treated with one of the treatments including active carbon treatment, far infrared light radiating treatment, and fragrance treatment.

2. The health insole as claimed in claim 1, further comprising a bottom photocatalyst layer bonded to the bottom surface of said bottom functional layer.

3. The health insole as claimed in claim 1, wherein said top photocatalyst layer is made of a material selected from the material group including high-strength titanium dioxide fibers, visible light-reactive photocatalyst paper, cloth, leather and synthetic resin.

4. The health insole as claimed in claim 1, wherein said high performance fibers are selected from one of the materials including nature fibers, corn fibers, acrylic fibers, nylon 6, and nylon 66.

5. The health insole as claimed in claim 1, wherein said nonwoven fabric is obtained from one of the materials including paper, cloth, leather, and synthetic resin.

6. The health insole as claimed in claim 1, wherein said active carbon has a grain or fiber-like shape.
